(19) United States
(12) Patent Application Publication
(10) Pub. No.: US 2016/0017401 A1
(43) Pub. Date: Jan. 21, 2016

(54) Method and apparatus for trapping and growing micro-organisms using pre-filter pads and similar pads

(71) Applicants: Geoffrey N. Roth, Goshen, IN (US); Jonathan N. Roth, Goshen, IN (US)

(72) Inventors: Geoffrey N. Roth, Goshen, IN (US); Jonathan N. Roth, Goshen, IN (US)

(21) Appl. No.: 14/120,968

(22) Filed: Jul. 15, 2014

(51) Int. Cl. C12Q 1/24

(52) U.S. Cl. CPC C12Q 1/24

(57) ABSTRACT

A method and apparatus for achieving optimum results of MF and/or Microtrap™ methods considering all of the basic elements that may be included in the MF and/or Microtrap™ apparatus with or without the need for a separate membrane filter and pad. In a major new addition to the classical MF and Microtrap™ approaches in the present invention, a different type of pad can be used to serve the function of both a pad and a membrane filter, (e.g. what is known in the industry as a “pre-filter”) through the use of fine mesh glass material for the pad, that material being non-toxic to the target micro-organisms (formed from fiberglass filaments, for example), with small openings (e.g. 0.3 to 0.7 micrometers). The pad may be pre-treated with growth media, preferably in dehydrated format, and formed of sufficient thickness to absorb and retain the sample fluid in which the target organisms are entrained. That fluid passing into the pad may, for example, function to activate the growth media so that it is available to the micro-organisms retained on the surface of the mesh.

### FIELD OF THE INVENTION

The present invention relates to methods and apparatus of examining samples of items contained within a fluid, especially for growing, maintaining, detecting, and/or examining biological entities, micro-organisms, such as bacteria and the like.

### BACKGROUND OF THE INVENTION

In the history of microbiology, there have been many detection methods and devices. The agar in a petri dish device and pour plate and streak plate method, and the membrane filter methods have been some of the most common and accepted methods. They typically use a container that can be used to support a usually water based growth and/or indicator medium, which is made available to a substance that can be used for support of individual colony forming units (CFUs) of microbes. When agar is used, colony forming units may form in the agar or on its surface. When membrane filter methods are used, colony forming units are usually on or part way in a membrane, which may be placed on an agar medium or on a pad carrying medium, where a pad is generally a flat permeable piece composed of CME or mixed cellulose esters that may be used to receive and contain and provide said medium. The microbes grow as colony forming units on or near the surface of the membrane.

An aspect of the present invention is considered to be an improvement upon prior microbiological Membrane Filter (“MF”) methods, such as those described in prior internet publications at http://www.pall.com/main/laboratory/literature-library-details.page?id=7290, http://www.rapidmicrobiology.com/test-method/theory-and-practice-of-microbiological-water-testing, and at http://edusanjalmicro.blogspot.com/2010/07/membrane-filter-technique.html. According to certain prior MF methods, a sample of the fluid to be tested (drinking water, for example) would be analyzed for the presence of bacteria or other organisms by passing a 100 mL sample, for example, through a porous membrane blocking passage through a funnel, that membrane having openings of, for example, approximately 0.45 micrometers in diameter. In certain applications, fluid can be passed through the membrane by the presence of a partial vacuum or air pressure differential through the openings of the funnel.

After the fluid passes through the membrane, the membrane (having trapped representative micro-organisms thereon) is removed from the funnel and placed onto a solid surface (which may be a layer of agar and/or a pad, typically in a petri dish), having a nutrient or growth media available therein to encourage growth of micro-organisms. The Petri dish is then incubated for an appropriate time and examined for the presence of colonies of micro-organisms, using a variety of conventional techniques.

However, certain official specifications for MF methods and apparatus are many times not sufficiently specific to avoid significant variation in the construction and/or materials of membrane filters by the various manufacturers of approved products. It has been found that there were significant differences in method results when using the various brands of membrane filters and/or pads used in such MF. Further, both the membrane filters and growth media pads used in the prior art are often composed of cellulose materials. It has been found by the applicants that when certain of those pads were used, there was slower detection of some micro-organisms. In addition, when using chromogenic and fluorogenic testing for micro-organisms, it has been found by the applicants that with certain of those cellulose pads during examination of the apparatus the ability to discriminate micro-organism colonies after incubation of the micro-organisms was reduced.

Accordingly, it is an object of the present invention to provide an improved method of and apparatus for trapping and growing micro-organisms, and the like, in order to facilitate detection some items. In addition, when using chromogenic and/or fluorogenic testing for microorganisms, it was found by the applicants that with certain of those pads during examination of the apparatus, the discrimination of microorganism colonies after incubation of the microorganisms was more difficult. Accordingly, it is an object of the present invention to provide an improved method of and apparatus for trapping microorganisms and the like, in order to facilitate detection of those items. Other objects include the provision of such detection apparatus and methods which:
a. are less expensive to use, more accurate, and produce faster results,
b. produce a greater volume of detectable micro-organism colonies within a given time period,
c. require less storage space prior to use,
d. provide more consistent results across various approved manufacturers,
e. have a longer shelf life,
f. have greater versatility in the nature and extent of the testing to be done, and
g. facilitate determination of the optimum types of membranes and pads to produce the best results.

### SUMMARY OF THE INVENTION

These and other objects of the present invention are obtained by providing a method and apparatus for achieving optimum results of MF and/or Microtrap™ methods considering all of the basic elements that may be included in the MF and/or Microtrap™ Apparatus (the filter type and brand and/or the pad type and its brand and composition, for example) with or without the need for a separate membrane filter and pad. In a major new addition to the classical MF and Microtrap™ approaches in the present invention, a different type of pad can be used to serve the function of both a pad and a membrane filter through the use of fine mesh glass material with small openings (e.g. 0.3 to 0.7 micrometers), that material being non-toxic to the target micro-organisms. The pad may be pre-treated with growth media, preferably in dehydrated format, and formed of sufficient thickness to absorb and retain the sample fluid in which the target organisms are entrained. That fluid passing into the pad may, for example, function to activate the growth media so that it is available to the micro-organisms retained on the surface of the pad. This is considered to be a major innovation or improvement to the MF Method.

Other objects, advantages, and novel features of the present invention will become readily apparent to those of skill in the art from the following detailed description and claims.

### BRIEF DESCRIPTION OF THE DRAWING

FIG. 1 shows a cross-sectional view of a representative apparatus for use with a preferred embodiment of the present invention. For illustrative clarity, in the figure the pad is shown to have significantly smaller dimensions that the width of the dish into which it is placed.

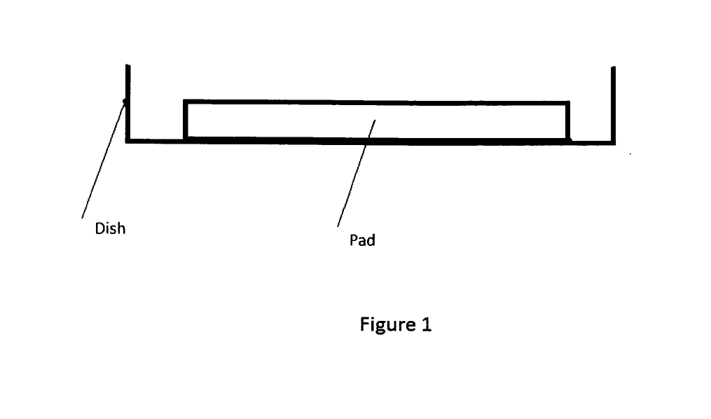

### DESCRIPTION OF PREFERRED EMBODIMENTS

For the purposes of this patent, a pad of the prior art was generally a flat permeable (e.g. cellulose) piece that may be used to receive and contain and provide medium. For this invention, a pad (now referred to as a “new” pad which may be comprised of materials chosen from a group comprising glass fibers, sintered plastic and other materials, can be used instead of, or substituted for, the traditionally used cellulose pads or other pads used in traditional membrane filter methods and/or Microtrap™ methods. This is thought to be the first report of this.

The same is true of the above mentioned “new” pad used as (in place of) a filter, instead of a micropore membrane, or a pad and a membrane filter used together. In cases of such improved or altered membrane filter methods and improved and altered Microtrap™ methods, better growth and/or recovery was detected, and the same was true when the methods used chromogenic substrates, where better color and color intensity were particularly detected. The above parts of the invention can be used with KwikCount™ also.

The new pad type may have various sized openings. In the above aspects, water and wet or dry media may be added in any order to the test container and/or conventional pad and/or “new pad”, and/or for conventional micropore membrane in any order and at the same time or not with the test sample.

In a preferred embodiment, the present invention is intended to be used with the similar types of apparatus and with similar types of methods as previously used with MF methods and Microtap™ methods except as follows. When the embodiment is a modification of the classical MF Method, there may be no need for a separate membrane filter, apart from a “new” pad. Thus, the steps of applying a larger sample of fluid to a membrane filter, allowing or drawing that fluid through the filter, collecting that fluid below the filter, and then removing the filter and applying it to a separate growth pad, are not needed. The extra equipment for those steps, such as a funnel, the separate membrane filter element, sterile forceps to move the filter to the growth pad, vacuum or pressure differential devices, etc., are not needed with this embodiment of the present invention.

Instead, with an embodiment of the present invention, a smaller quality of sample fluid, typically 1 mL (or on certain occasions 2 to 3 mL), can be applied directly to a “new” pad. The micro-organisms within that fluid are typically retained at or near the surface of the pad, while fluid itself goes into the pad. In preferred embodiments, fluid does not pass completely through the growth pad, but is retained in the pad. However, the present invention contemplates embodiments where some of the fluid actually passes out of the pad for collection elsewhere. Once the micro-organisms are deposited on the pad, the pad is subjected to whatever incubation or growth conducive treatment is usually employed with that type of micro-organism in prior MF and/or Microtrap™ techniques. Thereafter, the usual examination techniques may also be employed to detect and determine the micro-organism colony growth.

More specifically, a “new” pad of the present invention may be formed of a mesh of filaments which are not toxic to the target organisms. It also may be desirable that any filaments used in a “new” pad of the present invention do not exhibit any significant luminescence under ultraviolet lighting or other lighting used in a given micro-organism testing procedure. “New” pads may be made of materials chosen from a group comprising fiberglass, glass, quartz, and sintered plastic filaments in many instances. Examples of such commercially available products which are likely to work, as the “new” pad with embodiments of the present invention are “Fiber Glass Prefilters” types GA-40, GA-50, GA-55, and GA-75, offered for sale by Advantec MFS, Inc. (See www.advantecmfs.com).

Prior to receiving the fluid sample, a “new” pad of the present invention may be preferably treated with a nutrient or growth media. This can be done in a conventional manner, such as by adding a fluid broth onto the pad or applying a dusting of dehydrated media or other methods previously used to apply such media to traditional pads in MF and/or Microtrap™ methods, according to the needs for particular target micro-organisms being sought. Using dehydrated media, for example, allows pads to be more easily stored for longer periods of time until testing is desired.

When testing is to be done, the pads are preferably placed within a like container, such as a Petri dish or, film (e.g. Easygel Card™ and Petrifilm™). Often, it may be most convenient to have the pads dimensioned to substantially conform to the interior dimensions of that container. Afterwards, a sample of fluid to be tested, (e.g. about 1 mL in volume), may be applied to the top surface of the pad to be examined for the presence of certain target micro-organisms. When the fluid is applied to the pad, openings of the pad, (e.g. between 0.3 and 0.7 micrometers) are sufficient to allow fluid to enter into the pad while leaving target micro-organisms at or near the top surface of the pad. As fluid enters the pad, it may facilitate contact between the micro-organisms and the growth media which was previously placed on the pad or impregnated into the pad.

Thereafter, the pad is incubated or otherwise subjected to a growth inducing environment for the target micro-organisms in conventional manners or any other manner hereafter deemed appropriate for the desired testing. After whatever growth period is deemed appropriate for given micro-organisms, the pad device is examined for the presence of those micro-organisms, again in conventional manners or any other manner hereafter deemed appropriate for the desired testing.

With the present invention, it has been found that higher bacteria counts are possible within the same periods of incubation time, as compared to prior MF methods using traditional cellulose pads and traditional membrane filters. In addition, some aspects of the present invention have been found to allow higher visual discrimination (with or without ultraviolet lighting) of micro-organism colonies, as compared to prior MF methods using some traditional pads and some traditional membrane filters.

As an embodiment of this invention, traditional pads may be used with methods of this invention, but not every one of the features and/or advantages of the invention may be realized in this case.

In certain applications, some of the advantages of the present invention are available with otherwise conventional MF methods and/or methods of detecting micro-organisms where a membrane filter is used and then applied to the pad type of the present invention. In many of those applications, the pad type of the present invention or a different pad with similar properties superior to the prior traditional pad type permits the user to avoid the disadvantages of prior some traditional pads. Likewise, any membrane filter is preferred to be of a type to have been comparatively tested to ensure production of optimum results pertaining to growth, fluorescence or other factors. However, such applications may involve extra steps and equipment and, when some membrane filters (e.g. some comprised of cellulose) are used, they may not achieve a full extent of the benefits of embodiments of the present invention, as compared to methods of use described above.

Accordingly, the present invention has been described herein with respect to certain preferred embodiments, but the spirit and scope of this invention are limited only by the scope of the following claims.

What is claimed is:

1. A membrane filter method using a pad chosen from a group which comprises pads other than traditional pads as a pad, membrane, or both to allow improved results.

2. The method of claim 1 wherein any number of traditional and/or new pads may be used.

3. The method of claim 1 wherein the pad is chosen from a group comprising fiber glass, and sintered plastic.

4. A Microtrap™ method using a pad chosen from a group which comprises a pad other than the traditional pads as the pad, membrane, or both to allow improved results.

5. The method of claim 4 wherein any number of traditional and/or new pads may be used.

6. The method of claim 4 wherein the pad is chosen from a group comprising fiber glass and sintered plastic.

7. A method for trapping and growing micro-organisms using a pad, comprising:
applying growth media to said pad formed from material which is not toxic to the target micro-organisms, that pad being formed from a mesh of filament material,
applying a test sample of fluid to the pad, and
facilitating growth of the target micro-organisms on the pad and testing the pad thereafter for micro-organisms thereon.

8. The method according to claim 7 wherein the pad is formed from a material having spacing between 0.3 and 0.7 micrometers.

9. The method according to claim 7 wherein the pad is formed so as to retain therein substantially all of the fluid while separating the target micro-organisms originally entrained within the test sample to the be retained at or near the surface of the pad where the fluid was originally applied to the pad.

10. The method according to claim 7 wherein the pad is formed to allow the fluid to pass into the pad, leaving the target micro-organisms at or near the surface of the pad, and facilitates communication of the target micro-organism with the growth media on or within the pad.

11. The method according to claim 9 wherein the pad is formed from a mesh of filaments made from material which has no significant luminescence under the lighting used to determine the existence and extent of growth of micro-organisms on the pad.

12. The method according to claim 11 wherein the pad is formed from a material having filament spacing between 0.3 and 0.7 micrometers, and wherein the pad is formed so as to retain therein substantially all of the fluid while separating the target micro-organisms originally entrained within the test sample to the be retained at or near the surface of the pad where the fluid was originally applied to the pad, and wherein the pad is formed such that passage of the fluid into the pad facilitates communication of the target micro-organism with the growth media on or within the pad.

13. The method according to claim 12 wherein the pad is selected from a group of materials comprising fiberglass and sintered plastic.

14. The method according to claim 12 wherein the pad is formed from either fiberglass or sintered plastic.

15. The method according to claim 7 wherein this method is used in connection with a conventional MF method to replace use of a separate membrane filter and use of a cellulose growth pad.

16. The method according to claim 7 wherein this method is used in connection with a Microtrap™ method to replace use of a separate membrane filter and use of a cellulose growth pad.
